(19) United States
(12) Patent Application Publication
(10) Pub. No.: US 2023/0172532 A1
(43) Pub. Date: Jun. 8, 2023

(54) METHOD OF CALCULATING IN VIVO FORCE ON AN ANTERIOR CRUCIATE LIGAMENT

(71) Applicants: Griffith University, Nathan (AU); The University of Queensland, St Lucia (AU)

(72) Inventors: David Saxby, (AU); David Lloyd, (AU); Azadeh Nasseri, (AU); Hamid Khataee, (AU)

(21) Appl. No.: 17/768,999

(22) PCT Filed: Oct. 16, 2019
(86) PCT No.: PCT/AU2019/051129

§ 371 Date: Apr. 14, 2022

(51) Int. Cl. A61B 5/00; A61B 5/11; A61B 5/389; A61B 5/313; G16H 50/50

(52) U.S. Cl. CPC A61B 5/4533; A61B 5/4585; A61B 5/1124; A61B 5/389; A61B 5/313; G16H 50/50

(57) ABSTRACT

A method of calculating in vivo force on an anterior cruciate ligament (ACL) by measuring one or more biomechanical properties during a biomechanical screening task to obtain one or more biomechanical datum from the measured one or more biomechanical properties, and calculating a total load on an anterior cruciate ligament from an ACL force model using the one or more biomechanical datum as inputs to the ACL force model. The ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force interaction relationships among the sagittal-frontal (SF), sagittal-transverse (ST), and frontal-transverse (FT) planes, where j=SF, ST, FT.

### FIELD

The present disclosure relates to a method and system of calculating in vivo force on the anterior cruciate ligament of a subject without invasive techniques or procedures.

### BACKGROUND

Any references to methods, apparatus or documents of the prior art are not to be taken as constituting any evidence or admission that they formed, or form, part of the common general knowledge.

The anterior cruciate ligament (ACL) plays a crucial role in knee stability and is the most commonly injured knee ligament.

Although ACL loading patterns have been investigated under specific conditions (i.e., cadaver experiments, unvalidated models, or models without extensibility to new conditions), the interactions between knee loadings transmitted to the ACL remain unclear and inhibits efforts to prevent ACL injuries.

The ACL is the major intra-articular knee ligament and plays a key role in knee stability. Non-contact ACL ruptures are common and debilitating injuries, especially among sportspeople, and usually occur without contact between athletes. These non-contact ACL ruptures often load to serious long-term health consequences, such as early onset knee osteoarthritis.

Video analysis of non-contact ACL injuries and medical imaging of intra-articular injury patterns suggest that landing, change of direction, and pivoting motor tasks are associated with non-contact ACL injuries.

It is understood that external knee loads applied in three planes of motion (i.e. the sagittal plane, the frontal plane and the transverse plane) contribute to ACL rupture and injury. However, the mechanisms by which external knee loads are transmitted to the ACL through the interaction of muscles, contacting articular bodies, and other soft tissues during dynamic motor tasks remains unresolved.

Some earlier studies have modelled ACL force (i.e. the force applied to the ACL) but were limited by simplistic loading conditions, have not been able to predict the experimentally observed ACL forces accurately, or were developed based on limited sample data. For example, some previous models assume that ACL force in response to multi-planer external knee loads is equal to the sum of forces exerted by multiple independent uni-planar loads. However, experiments have provided evidence that ACL force is not defined by a pure summation of the forces in each plane.

Some earlier studies use explicit representations of the ACL within whole-body anatomical models, and mechanical optimisation to derive muscle forces. However, these approaches have two serious limitations. First, explicit representation of the ACL requires subject-specific anatomical information of the knee and plethora articular tissues (i.e. cartilages, menisci, and capsule), as well as mechanical properties of ACL, other knee ligaments, and articular tissues. Acquiring these data is non-trivial as there is no accepted non-invasive method to measure subject-specific mechanical properties of native knee tissues.

Moreover, if there were, such non-invasive techniques would likely be resource intensive, thus limiting suitability for clinical applications.

Second, mechanical optimisation typically employed to determine muscles force is unlikely to predict several empirically observed features of muscle coordination, and is insensitive to task-specific control objectives, and pathology.

Thus, there remains a need to identify a model to quantify ACL force on a subject-specific basis without invasive techniques or procedures.

### SUMMARY OF INVENTION

In one form, there is provided a method of calculating in vivo force on an anterior cruciate ligament (ACL), the method comprising:

measuring one or more biomechanical properties during a biomechanical screening task to obtain one or more biomechanical datum from the measured one or more biomechanical properties; and

calculating a total load on an anterior cruciate ligament from an ACL force model using the one or more biomechanical datum as inputs to the ACL force model.

In an embodiment, a neuromusculoskeletal model is calculated from the one or more biomechanical datum.

In an embodiment, the method comprises constructing a neuromusculoskeletal model from the one or more biomechanical datum.

In an embodiment, the ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT.

In an embodiment, F_{ACL}^{sag}=a₁F_{AD}θ²+a₂F_{AD}θ+a₃F_{AD}+a₄e^{(a}^{₅}^{F}^{_{AD}}^{+a}^{₆}^{θ)}, wherein a₁=1.8×10⁻⁴±5.6×10⁻⁷, a₂=0.02±0.1×10⁻⁴, a₃=1.16±0.005, a₄=32.15±0.02, a₅=3.9×10⁻⁵±1.8×10⁻⁴, and a₆=−0.022±2.3×10⁻⁵, F_{AD} is anterior force drawer and θ is knee flexion angle.

In an embodiment.

$${F}_{ACL}^{front} = \{ \begin{matrix}{b}_{1} {M}_{var} {θ}^{2} + {b}_{2} {M}_{var} θ + {b}_{3} {M}_{var} + {b}_{4} {e}^{( {b}_{5} θ )} , if varus \\ {c}_{1} {M}_{valg} {θ}^{2} + {c}_{2} {M}_{valg} θ + {c}_{3} {M}_{valg} + {c}_{4} {e}^{( {c}_{5} θ )} + {c}_{6} {e}^{( {c}_{7} {M}_{valg} )} , if valgus\end{matrix} ,$$

wherein b₁=−0.0014±0.1×10⁻³, b₂=0.18±0.01, b₃=−6.8±0.21, b₄=23.85±2.03, b₅=−0.14±0.03 for varus moment; and c₁=−0.001±3.6×10⁻⁸, c₂=0.08±3.2×10⁻⁶, c₃=2.5±5.2×10⁻⁵, c₄=−3.3±0.6×10⁻⁵, c₅=−0.04±6.7×10⁻⁷, c₆=29.3±0.3×10⁻⁴, and c₇=0.02±3×10⁻⁷ for valgus moment, Mᵥₐᵣ is knee varus moment, M_{valg} is knee valgus moment and θ is knee flexion angle.

In an embodiment,

$${F}_{ACL}^{trans} = \{ \begin{matrix}{m}_{1} {M}_{IR} {θ}^{2} + {m}_{2} {M}_{IR} θ + {m}_{3} {M}_{IR} + {m}_{4} {e}^{( {m}_{5} θ )} , if internal rotation \\ {n}_{1} {M}_{E R} {θ}^{2} + {n}_{2} {M}_{E R} θ + {n}_{3} {M}_{E R} + {n}_{4} {e}^{( {n}_{5} θ )} , if external rotation\end{matrix} ,$$

wherein m₁=−0.005±2.4×10⁻⁷, m₂=0.63±0.2×10⁻⁴, m₃=−20.03±3.8×10⁻³, m₄=36.6±3.4×10⁻², m₅=−0.04±7.1×10⁻⁶ for internal rotation moment; and n₁=0.001±2×10⁻³, n₂=−0.16±0.02, n₃=7.8±0.4, n₄=23.3±2.5, n₅=−0.06±0.01 for external rotation moment, M_{IR} is internal rotation moment of the knee, M_{ER} is external rotation moment of the knee and θ is knee flexion angle.

In an embodiment,

$$C {T}_{SF} = \{ \begin{matrix}{p}_{1} {F}_{ACL}^{front} {e}^{( {p}_{2} {F}_{ACL}^{sag} )} + {p}_{3} θ {e}^{( {p}_{4} θ )} , if varus \\ {q}_{1} {e}^{( {q}_{2} {F}_{ACL}^{front} )} + {q}_{3} θ {e}^{( {q}_{4} θ )} , if valgus\end{matrix}$$

Where p₁=−0.84±8.2×10⁻⁶, p₂=−0.004±6.9×10⁻⁸, p₃=2.9±1.3×10⁻⁵, and p₄=−0.041±1.02×10⁻⁷ for varus moment; and q₁=39.1±1.4×10⁻⁴, q₂=0.002±9.7×10⁻¹⁰, q₃=8.7±1.9×10⁻⁶, and q₄=−0.03±3.4×10⁻⁹ for valgus moment.

In an embodiment.

$$C {T}_{S T} = \{ \begin{matrix}{v}_{1} {F}_{ACL}^{sag} {F}_{ACL}^{trans} + {v}_{2} {e}^{( {v}_{3} θ )} , if internal rotation \\ {w}_{1} {F}_{ACL}^{trans} {e}^{( {w}_{2} {F}_{ACL}^{sag} )} + {w}_{3} {e}^{( {w}_{4} θ )} , if external rotation\end{matrix}$$

Where v₁=6.8×10⁻³±1.1×10⁻⁹, v₂=−32.2±3.6×10⁻³, and v₃=0.01±1.8×10⁻⁷ for internal rotation; and w₁=−0.81±2.8×10⁻⁶, w₂=−0.003±1.3×10⁻⁷, w₃=−67.9±4.3×10⁻⁴, and w₄=−0.001±1.8×10⁻⁷ for external rotation.

In an embodiment, CT_{FT}=0.

In an embodiment, the method comprises calculating a load on an anterior cruciate ligament in each of three planes of motion.

In an embodiment, the one or more biomechanical datum are measured in three-dimensions.

In an embodiment, the three dimensions are defined by three planes of motion. In an embodiment, the three planes of motion comprise:

a sagittal plane;

a transverse plane; and

a frontal plane.

In another form, there is provided a method of calculating in vivo force on an anterior cruciate ligament (ACL), the method comprising:

calculating a total load on an anterior cruciate ligament from an ACL force model defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT.

In an embodiment, the method comprises generating a graphical representation of the calculated total load on a display of a computer.

In yet another form, there is provided a method of operating one or more electronic processors to calculate in vivo force on an anterior cruciate ligament, the method comprising:

acquiring one or more biomechanical datum in an electronic storage assembly accessible to said processors; and

calculating a total load on an anterior cruciate ligament from an ACL force model combined with the one or more biomechanical datum, wherein the ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT.

In yet another form, there is provided a method of operating one or more electronic processors to calculate in vivo force on an anterior cruciate ligament, the method comprising:

inputting one or more biomechanical datum to an ACL force model; and

calculating a total load on an anterior cruciate ligament from the ACL force model combined from the one or more inputted biomechanical datum, wherein the ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT.

In yet another form, there is provided a software program configured to execute an ACL force model, wherein the software program is operable to:

receive one or more biomechanical datum as data inputs; and

calculate, via operation of one or more electronic processors, in vivo force on an anterior cruciate ligament using the ACL force model and the one or more biomechanical datum as data inputs to the ACL force model.

In an embodiment, the software program receives the one or more biomechanical datum through a graphical user interface on a display of a computer having the software program installed thereon. Alternatively, or in addition, the software program receives the one or more biomechanical datum as an input file that is uploaded to the software program from a database or memory of the computer having the software programmed installed thereon.

In another form, there is provided a system for calculating an in vivo force on an anterior cruciate ligament (ACL), the system comprising:

a biomechanical screening system configured for a subject to perform a dynamic motor task, the biomechanical screening system comprising one or more biomechanical property monitoring apparatus for monitoring one or more biomechanical properties of the subject performing the dynamic motor task, wherein the one or more biomechanical monitoring apparatus generate one or more biomechanical datum; and
a computer having one or more electronics and a software product installed thereon, the software product being configured to operate the one or more electronic processors of the computer to calculate in vivo force on an anterior cruciate ligament (ACL) from an ACL force model by:
   receiving the one or more biomechanical datum as data inputs; and
   calculating, via operation of the one or more electronic processors, in vivo force on an anterior cruciate ligament using the ACL force model and the data inputs from the one or more biomechanical datum as inputs to the ACL force model.

In an embodiment, the dynamic motor task comprises a drop-landing test.

In an embodiment, the one or more biomechanical property monitoring apparatus of the biomechanical screening system comprise a motion capture system. In an embodiment, the motion capture system comprises one or more of: a plurality of inertial measurement units; an electromagnetic measurement system; and Artificial Intelligence based system.

In an embodiment, the one or more biomechanical property monitoring apparatus of the biomechanical screening system comprise at least one of the following:

at least one electromyograph (EMG) sensors for attaching to the subject;

a motion capture system comprising a plurality of motion capture cameras and a plurality of retroreflective markers for attaching to the subject, wherein the plurality of motion capture cameras are configured to track the retroreflective markers; and

at least one ground embedded force platform configured to measure three-dimensional ground reaction loads of the subject.

In an embodiment, marker trajectories of the retroreflective markers are filtered by a second-order, zero-lag Butterworth filter having a low-pass cut-off frequency of 6 Hz.

In an embodiment, ground reaction data from the ground embedded force platform is filtered by a second-order, zero-lag Butterworth filter having a low-pass cut-off frequency of 6 Hz.

In an embodiment, signals from the EMG sensors are filtered by a band-pass filter (between 30-300 HZ), full-wave rectified, and smoothed with a second-order Butterworth low-pass filter with a cut-off frequency of 6 HZ generating a plurality of EMG linear envelopes. In an embodiment, the EMG linear envelopes are normalised to the maximum linear envelope value of a corresponding muscle.

In another form, there is provided a method for calculating an in vivo force on an anterior cruciate ligament (ACL), the method comprising:

monitoring one or more biomechanical properties of a subject performing a dynamic motor task;

generating one or more biomechanical datum from the monitoring of the one or more biomechanical properties of the subject performing the dynamic motor task;

receiving the one or more biomechanical datum as data inputs to a computer implemented ACL force model for calculating in vivo force on an anterior cruciate ligament;

calculating in vivo force on an anterior cruciate ligament of the subject performing the dynamic motor task from the computer implemented ACL force model, wherein the ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT.

In another form, there is provided a method for creating a computer model of in vivo force on an anterior cruciate ligament (ACL) of a subject, the method comprising:

monitoring one or more biomechanical properties of a subject performing a dynamic motor task, wherein the subject is unshod;

generating a first set of one or more biomechanical datum from the monitoring of the one or more biomechanical properties of the subject performing the dynamic motor task;

receiving the first set of one or more biomechanical datum as data inputs to a computer implemented ACL force model for calculating in vivo force on an anterior cruciate ligament;

calculating a first in vivo force on an anterior cruciate ligament of the subject performing the dynamic motor task from the computer implemented ACL force model, wherein the ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT;

monitoring one or more biomechanical properties of the subject performing the dynamic motor task, wherein the subject is shod;

generating a second set of one or more biomechanical datum from the monitoring of the one or more biomechanical properties of the subject performing the dynamic motor task;

receiving the second set of one or more biomechanical datum as data inputs to a computer implemented ACL force model for calculating in vivo force on an anterior cruciate ligament;

calculating a second in vivo force on an anterior cruciate ligament of the subject performing the dynamic motor task from the computer implemented ACL force model, wherein the ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT.

In an embodiment, the method further comprises calculating a difference between the first in vivo force on the anterior cruciate ligament of the subject performing the dynamic motor task and the second in vivo force on the anterior cruciate ligament of the subject performing the dynamic motor task.

In another form, there is provided a method for creating a computer model of in vivo force on an anterior cruciate ligament (ACL) of a subject performing a dynamic motor task wearing different pairs of shoes, the method comprising:

monitoring one or more biomechanical properties of a subject performing a dynamic motor task, wherein the subject is wearing a first pair of shoes;

generating a first set of one or more biomechanical datum from the monitoring of the one or more biomechanical properties of the subject performing the dynamic motor task;

receiving the first set of one or more biomechanical datum as data inputs to a computer implemented ACL force model for calculating in vivo force on an anterior cruciate ligament;

calculating a first in vivo force on an anterior cruciate ligament of the subject performing the dynamic motor task from the computer implemented ACL force model, wherein the ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT;

monitoring one or more biomechanical properties of the subject performing the dynamic motor task, wherein the subject is wearing a second pair of shoes;

generating a second set of one or more biomechanical datum from the monitoring of the one or more biomechanical properties of the subject performing the dynamic motor task;

receiving the second set of one or more biomechanical datum as data inputs to a computer implemented ACL force model for calculating in vivo force on an anterior cruciate ligament;

calculating a second in vivo force on an anterior cruciate ligament of the subject performing the dynamic motor task from the computer implemented ACL force model, wherein the ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT.

Further features and advantages of the present invention will become apparent from the following detailed description.

### BRIEF DESCRIPTION OF THE DRAWINGS

Embodiments in accordance with the present disclosure will be described, by way of example, in the following Detailed Description of Preferred Embodiments, which provides sufficient information for those skilled in the art to perform the invention. The Detailed Description of Preferred Embodiments is not to be regarded as limiting the scope of the preceding Summary section in any way. The Detailed Description will make reference to the accompanying drawings, by way of example, in which:

FIG. 1 illustrates knee loading in the three planes of motion;

FIG. 2 illustrates a flowchart of steps of a method for calculating ACL force from an ACL force model in accordance with an embodiment of the invention;

FIG. 3 illustrates a biomechanical screening system and a subject performing a dynamic motor task for acquiring biomechanical data;

FIG. 4 is a block diagram of a computational apparatus in the form of a computer that is specially programmed to calculate in vivo ACL force from an ACL force model;

FIG. 5 illustrates a series of graphs modelling uni-planar ACL forces;

FIG. 6 illustrates a series of graphs modelling total ACL force vs. knee flexion;

FIG. 7 illustrates a series of graphs demonstrating validation of the ACL force model;

FIG. 8 illustrates a series of graphs modelling uni-planar ACL forces across the stance-phase of a drop-landing task; and

FIG. 9 illustrates a graph modelling total ACL force and uni-planar ACL forces across the stance-phase of a drop-landing task.

### DETAILED DESCRIPTION OF PREFERRED EMBODIMENTS

With reference to the accompanying drawings, embodiments of a method for calculating in vivo force on the anterior cruciate ligament of a subject without invasive techniques or procedures in accordance with the present disclosure will now be described.

The present disclosure relates to a model (typically a computer implemented model) for quantifying ACL force. That is, a computation model which accurately and simply calculates the force that an anterior cruciate ligament of a living subject is subject to during a dynamic motor task without the need for invasive procedures or techniques.

The inventors have found that total ACL force F_{ACL} is not the simple summation of the uni-planar ACL forces (i.e. F_{ACL}≠F_{ACL}^{sag}+F_{ACL}^{front}+F_{ACL}^{trans}) in the respective sagittal, frontal and transverse planes. Indeed, the inventors have found that pure summation results in over- and under-estimation of F_{AIL}, depending on knee flexion angle θ and external loading (F_{AD}, Mᵥₐᵣ, M_{valg}, M_{IR}, M_{ER}) magnitudes. The implication is that interactions between multiple uni-planar ACL forces influence the total force transmitted to the ACL. Thus, the inventors have found that the total ACL force can be modelled by the following equation (Equation (1)):

F_{ACL}=F_{ACL}^{sag}+F_{ACL}^{front}+F_{ACL}^{trans}+ΣⱼCTⱼ

Where F_{ACL}^{sag} is ACL force in the sagittal plane, F_{ACL}^{front} is ACL force in the frontal plane, F_{ACL}^{trans} is ACL force in the transverse plane, and CTⱼ for j=SF, ST, FT represents ACL force relationships in the sagittal-frontal (SF) plane, the sagittal-transverse (ST) plane, and the frontal-transverse (FT) plane.

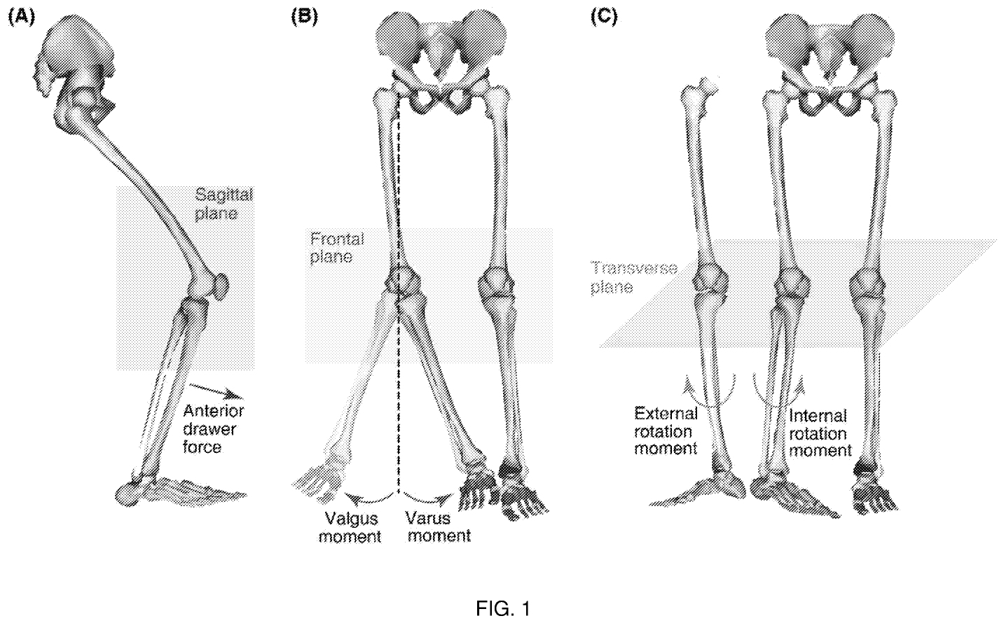

Exemplary illustrations of knee loading in each plane can be seen in FIGS. 1(A)-(C). Specifically, FIG. 1(A) illustrates knee loading in the sagittal plane, FIG. 1(B) illustrates knee loading in the frontal plane and FIG. 1(C) illustrates knee loading in the transverse plane.

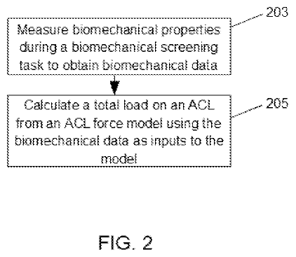

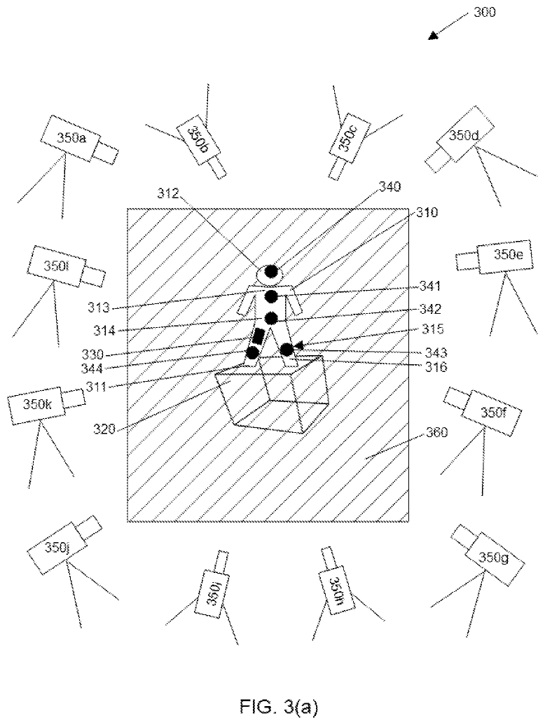

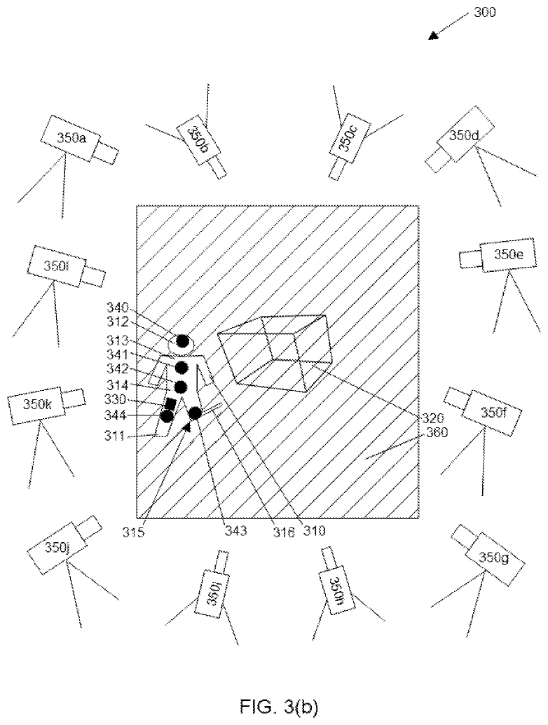

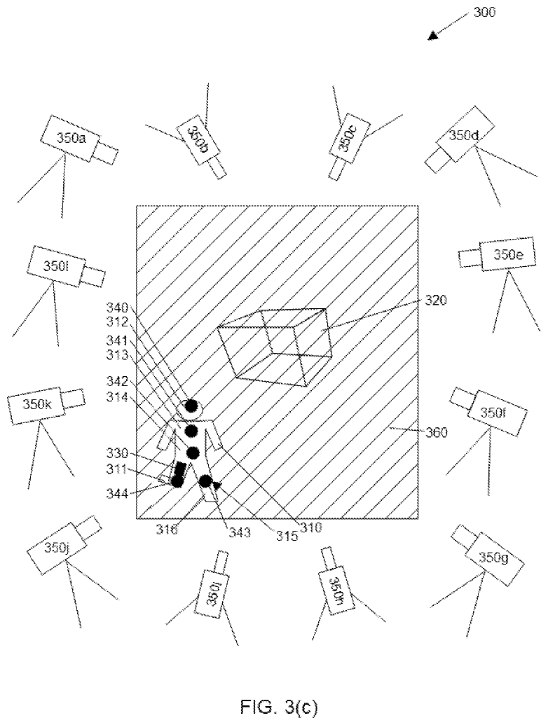

Referring now to FIG. 2, there is illustrated a block diagram of an exemplary method (that may be implemented in computer system 33 described below, for example) of calculating in vivo force on an anterior cruciate ligament (ACL). In the illustrated embodiment, in function step 203 of method 200, one or more biomechanical properties are measured from a biomechanical screening task. From these measurements, one or more biomechanical datum can be obtained. An example of this is shown in FIGS. 3a-c and will described below.

Turning to function step 205, the method 200 includes calculating a total load on an anterior cruciate ligament from the ACL force model (defined by F_{ACL}=F_{ACL}^{sag}+F_{ACL}^{front}+F_{ACL}^{trans}+ΣⱼCTⱼ) using the one or more biomechanical datum as inputs to the mathematical model.

With reference now to FIG. 3(a)-(c), an example of the measurement of biomechanical properties of a subject performing a biomechanical task to obtain biomechanical data will be described.

Referring first to FIG. 3(a), there is shown a biomechanical screening system 300 to allow a biomechanical screening task to be performed. Performing the biomechanical screening task using biomechanical screening system 300 is subject 310, having a number of sensors attached to their body. As can be seen, subject 310 is standing on a box 320, the purpose of which will be described later.

A number of wireless EMG sensors 330, shown as a strip, are secured over the rectus femoris, vastus lateralis, vastus medialis, tibialis anterior, lateral gastrocnemius, medial gastrocnemius, lateral hamstrings, and medial hamstring muscles on the landing leg 311 of the subject 310. In the present example, the EMG sensors 330 are placed only on the landing leg 311 and the signals of the EMG sensors 330 are measured at 2400 Hz. However, EMG sensors could be placed on both legs (i.e. landing and non-landing) to make comparisons between sides in future applications. Moreover, the signals of the EMG sensors 330 could be measured at any rate above 1000 Hz.

Retroreflective markers 340-344 are also respectively attached to the subject 310 on their head 312, trunk 313, pelvis 314, and lower body 315 including the thighs, shanks, and feet on both the non-landing leg 316 and landing leg 311. These retroreflective markers 340-344 are monitored by a motion capture system comprising 12 motion capture cameras 350a-l (hereinafter referred to collectively as motion capture cameras 350) arranged around the subject 310 to capture the 3D position of the retroreflective markers 340-344 and measure kinematic data collected at 120 Hz (or any rate greater than 100 Hz). While a motion capture camera system has been described, it will be appreciated that any motion capture system can be used. For example, a motion capture system including one or more of inertial measurements, electromagnetic systems or Artificial Intelligence based systems could be used to capture motion data.

The biomechanical screening system 300 also includes a ground-embedded force platform 360 which measures three-dimensional ground reaction loads at 2400 Hz. It should be appreciated that the three-dimensional ground reaction loads may be measured anywhere between 1000 Hz and 2400 Hz.

The biomechanical screening task to be performed by subject 310 using biomechanical screening system 300 involves the subject 310 hopping down from the box 320 (set at 30% of lower limb length of the subject 310) to land on one leg (landing leg 311) immediately followed by a 90° lateral jump landing on their opposite leg (non-landing leg 315).

Marker trajectories of the retroreflective markers 340-344 and ground reaction data from the ground embedded force platform 360 are filtered using a second-order, zero-lag Butterworth filter, with a low-pass cut-off frequency of 6 Hz.

The EMG data from the wireless EMG sensors 330 is band-pass filtered (between 30-300 HZ), full-wave rectified, and smoothed with a second-order Butterworth low-pass filter with a cut-off frequency of 6 HZ to produce linear envelopes. The EMG linear envelopes are then normalised to the maximum linear envelope value of the corresponding muscle from all available motion trials. These trials can include dedicate maximum effort contractions performed isometrically or dynamically.

This filtering described above provides biomechanical data that can be input into the ACL force model to calculate in vivo ACL loads.

In use, a subject may wear a pair of shoes thought to lower ACL force for the wearer as compared to another type of shoe or unshod condition. The subject would first perform the drop-landing test described above unshod and then again wearing the pair of shoes. In each case, the biomechanical screening system 300 would monitor the subject to determine the relevant knee kinematic and kinetic data, and muscle data for the instance of the test.

Subsequently, the relevant data would be input into the ACL force model to calculate total in vivo ACL force F_{ACL} for each test (i.e. unshod and shod). The different outputs of total in vivo ACL force can then be compared and contrasted and, importantly, used to scientifically and empirically verify that the pair of shoes (or any other product designed or claiming to reduce ACL force or assist the ACL) achieves what is being claimed by the designer.

In another example, the ACL force model may be used to calculate ACL force during use of training or gym equipment. In the same manner as the example described, a subject can be monitored during use of training equipment to thereby calculate the ACL force during use. These calculations can then be used to assess and advise users who may be rehabilitating after an injury and who cannot exceed certain loads during the rehabilitation process. In this regard, the ACL force model is particularly useful as a rehabilitation and injury prevention tool.

In addition, the ACL force model can be used to study different loads experienced by the ACL during different movements and exercises.

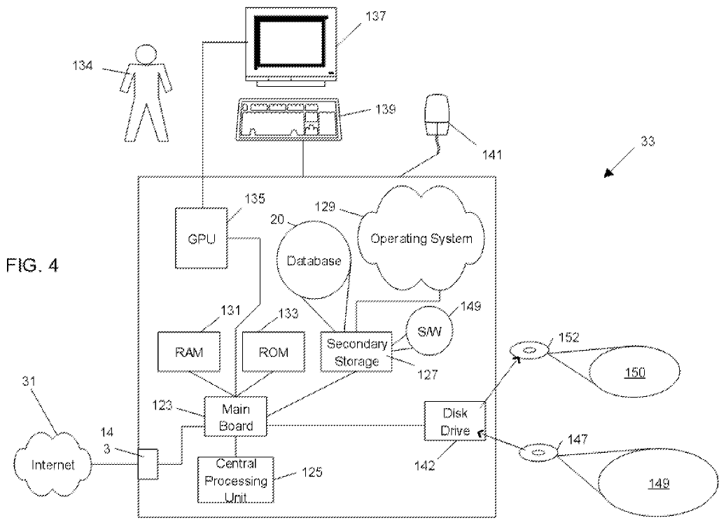

Referring now to FIG. 4, there is shown a block diagram of an exemplary computer system 33 for carrying out a method, such as method 200 described above, according to an embodiment of the invention that will be described.

The computer system 33 includes a main board 123 which includes circuitry for powering and interfacing to at least one on-board Central Processing Unit (CPU) 125. The one or more on-board processor(s) 125 may comprise two or more discrete processors or processor with multiple processing cores.

The main board 123 acts as an interface between CPU 125 and secondary memory storage 127. The secondary memory storage 127 may comprise one or more optical or magnetic, or solid state, drives. The secondary memory storage 127 stores instructions for an operating system 129.

The main board 123 includes busses by which the CPU is able to communicate with random access memory (RAM) 131, read only memory (ROM) 133 and various peripheral circuits. The ROM 133 typically stores instructions for a Basic Input Output System (BIOS) which the CPU 125 accesses upon start up and which prepares the CPU 125 for loading of the operating system 129.

The main board 123 also interfaces with a graphics processor unit (GPU) 135. It will be understood that in some systems the graphics processor unit 135 is integrated into the main board 123. The GPU 135 drives a display 137 which includes a rectangular screen comprising an array of pixels.

The main board 123 will typically include a communications adapter, for example a LAN adapter or a modem, either wired or wireless, that is able to put the computer system 33 in data communication with a computer network such as the Internet 31 via port 143.

A user 134 of the computer system 33 may interface with it by means of a keyboard 139, a mouse 141 and the display 137.

The computer system 33 automatically, via programming, commands the operating system 129 to load software product 149 which contains instructions for the computer system 33 to perform ACL force model calculations based on biomechanical data collected from biomechanical screenings (which will be explained in more detail below) by operation of CPU 125 and, in some embodiments, GPU 135. The calculations performed by software product 149 in combination with the CPU 125 may be stored in memory (as discussed above) or output on the display 137 in a graphical manner for immediate (i.e. real-time) consideration by the user 134.

The biomechanical data may be input by one of the interface mechanisms of the computer system 33 such as the keyboard 139, mouse 141 and display 137. The software product 149 may be provided as tangible instructions borne upon a computer readable media such as an optical disk 147 for reading by a disk reader/writer 142. Alternatively, the software product 149 might also be downloaded via port 143 from a remote data source via data network 145.

Software product 149 may also include instructions to read biomechanical data, which are variable inputs for the ACL force model, from secondary memory storage 127. Alternatively, or in addition, the software product 149 may also includes instructions to establish a database 20 which includes of the all ACL force model calculations and data that is generated from the calculations. Alternatively, the ACL force model data may be stored in another data storage arrangement that is accessible to computer system 33.

The methods and systems described above use the ACL force model F_{ACL}=F_{ACL}^{sag}+F_{ACL}^{front}+F_{ACL}^{trans}+ΣⱼCTⱼ derived by the inventors to accurately predict in vivo ACL loads in dynamic motor tasks. What follows is a description and explanation of the manner in which Equation (1) was derived and validated.

In quantifying total ACL force (F_{ACL}), the resultant ACL forces from external knee loading in three planes of motions (i.e. sagittal, frontal and transverse) were modelled.

A set of algebraic equations were fitted to experimental data [19-21], which measures resultant ACL force across knee flexion angles 0-45° in the presence of specific external knee loads.

In the sagittal plane, ACL force F_{ACL}^{sag} is modelled as a function of knee anterior drawer force F_{AD} and knee flexion angle θ by fitting Equation (2), below, to data [19-21]:

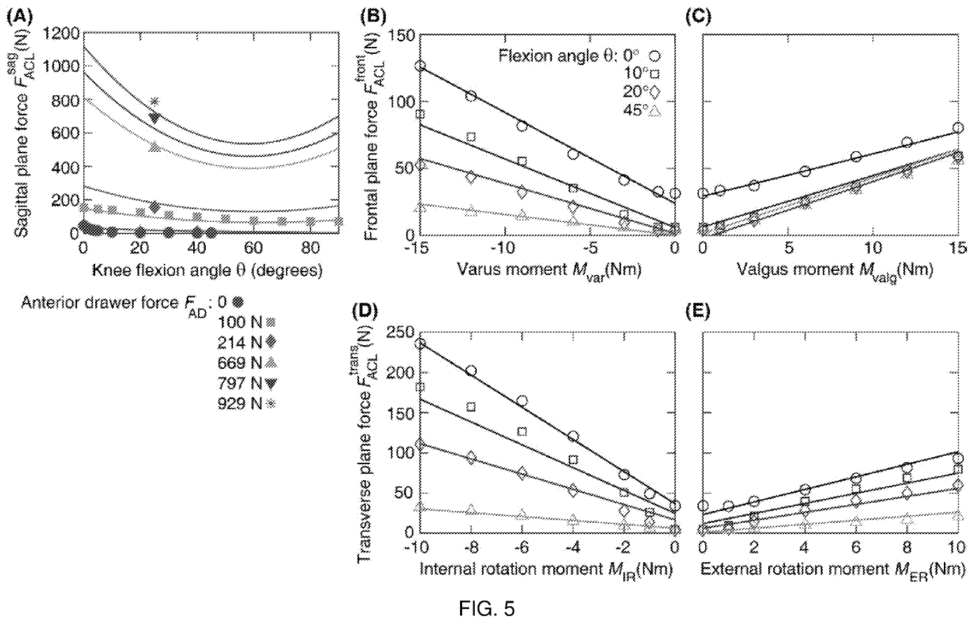

F_{ACL}^{sag}=a₁F_{AD}θ²+a₂F_{AD}θ+a₃F_{AD}+a₄e^{(a}^{_{S}}^{F}^{_{AD}}^{+a}^{₆}^{θ)}

where fit parameters are a₁=1.8×10⁻⁴±5.6×10⁻⁷, a₂=0.02±0.1×10⁻⁴, a₃=1.16±0.005, a₄=32.15±0.02, a₅=3.9×10⁻⁵±1.8×10⁻⁴, and a₆=−0.022±2.3×10⁻⁵ (see FIGS. 1(A) and 5(A)). Parameters values correspond to mean±standard error.

In the frontal plane, ACL force F_{ACL}^{front} is modelled as a function of knee varus or valgus moment (Mᵥₐᵣ or M_{valg}) and knee flexion angle θ by fitting Equation (3), below, to data [20]:

$${F}_{ACL}^{front} = \{ \begin{matrix}{b}_{1} {M}_{var} {θ}^{2} + {b}_{2} {M}_{var} θ + {b}_{3} {M}_{var} + {b}_{4} {e}^{( {b}_{5} θ )} , if varus \\ {c}_{1} {M}_{valg} {θ}^{2} + {c}_{2} {M}_{valg} θ + {c}_{3} {M}_{valg} + {c}_{4} {e}^{( {c}_{5} θ )} + {c}_{6} {e}^{( {c}_{7} {M}_{valg} )} , if valgus\end{matrix}$$

where fit parameters are b₁=−0.0014±0.1×10⁻³, b₂=0.18±0.01, b₃=−6.8±0.21, b₄=23.85±2.03, b₅=−0.14±0.03 for varus moment; and c₁=−0.001±3.6×10⁻⁸, c₂=0.08±3.2×10⁻⁶, c₃=2.5±5.2×10⁻⁵, c₄=−3.3±0.6×10⁻⁵, c₅=−0.04±6.7×10⁻⁷, c₆=29.3±0.3×10⁻⁴, and c₇=0.02±3×10⁻⁷ for valgus moment (see FIGS. 1(B) and 5(B), 5(C)).

In the transverse plane, ACL force F_{ACL}^{trans} was modelled as a function of an internal rotation moment of the knee (M_{IR}) or an external rotation moment of the knee (M_{ER}) and knee flexion angle θ by fitting Equation (4), below, to data [20]:

$${F}_{ACL}^{trans} = \{ \begin{matrix}{m}_{1} {M}_{IR} {θ}^{2} + {m}_{2} {M}_{IR} θ + {m}_{3} {M}_{IR} + {m}_{4} {e}^{( {m}_{5} θ )} , if internal rotation \\ {n}_{1} {M}_{E R} {θ}^{2} + {n}_{2} {M}_{E R} θ + {n}_{3} {M}_{E R} + {n}_{4} {e}^{( {n}_{5} θ )} , if external rotation\end{matrix}$$

where fit parameters are m₁=−0.005±2.4×10⁻⁷, m₂=0.63±0.2×10⁻⁴, m₃=−20.03±3.8×10⁻³, m₄=36.6±3.4×10⁻², m₅=−0.04±7.1×10⁻⁶ for internal rotation moment; and n₁=0.001±2×10⁻³, n₂=−0.16±0.02, n₃=7.8±0.4, n₄=23.3±2.5, n₅=−0.06±0.01 for external rotation moment (see FIGS. 1(C) and 5(D), 5(E)).

The cross-terms CTⱼ (where CTⱼ for j=SF, ST, FT represent ACL force relationships in the sagittal-frontal (SF) plane, the sagittal-transverse (ST) plane, and the frontal-transverse (FT) plane) are formulated by curve fitting to multi-planar ACL force data from [19, 21]. The inventors used all data for [19] and an eleven data point subset of the data from [21] which covers different loading magnitudes (i.e. from low to high) through each plane of motion (i.e. sagittal, frontal and transverse). This ensures that the model is developed by considering the entire range of the experimentally measured loads in every plane of motion as well as the combinations of these loads.

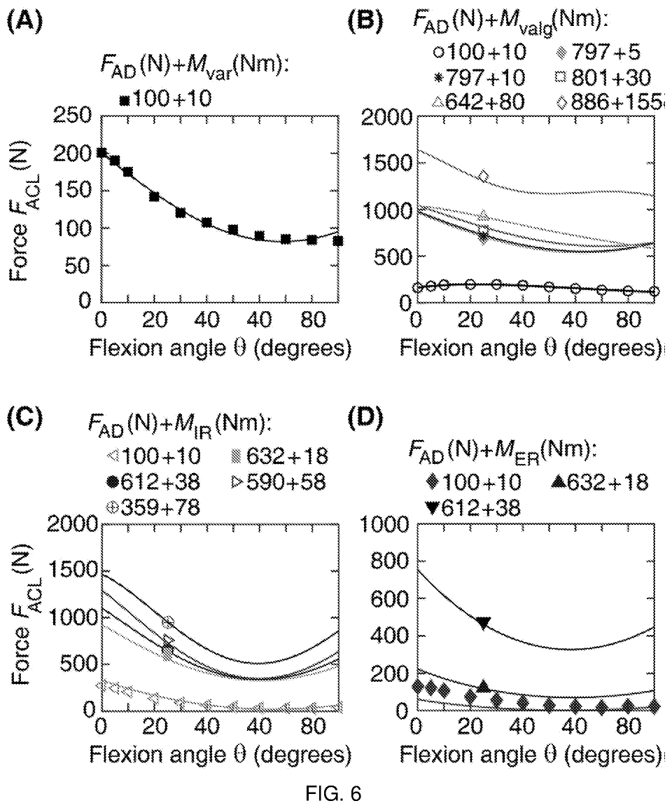

The interactions between sagittal and frontal planes (SF cross-terms) are found to be modelled by Equation (5), which is:

$$C {T}_{SF} = \{ \begin{matrix}{p}_{1} {F}_{ACL}^{front} {e}^{( {p}_{2} {F}_{ACL}^{sag} )} + {p}_{3} θ {e}^{( {p}_{4} θ )} , if varus \\ {q}_{1} {e}^{( {q}_{2} {F}_{ACL}^{front} )} + {q}_{3} θ {e}^{( {q}_{4} θ )} , if valgus\end{matrix}$$

Where p₁=−0.84±8.2×10⁻⁶, p₂=−0.004±6.9×10⁻⁸, p₃=2.9±1.3×10⁻⁵, and p₄=−0.041±1.02×10⁻⁷ for varus moment; and q₁=39.1±1.4×10⁻⁴, q₂=0.002±9.7×10¹⁰, q₃=8.7±1.9×10⁻⁶, and q₄=−0.03±3.4×10⁻⁹ for valgus moment (see FIGS. 6(A), 6(B)).

The interactions between sagittal and transverse planes (ST cross-terms) are found to be modelled by Equation (6), which is:

$$C {T}_{S T} = \{ \begin{matrix}{v}_{1} {F}_{ACL}^{sag} {F}_{ACL}^{trans} + {v}_{2} {e}^{( {v}_{3} θ )} , if internal rotation \\ {w}_{1} {F}_{ACL}^{trans} {e}^{( {w}_{2} {F}_{ACL}^{sag} )} + {w}_{3} {e}^{( {w}_{4} θ )} , if external rotation\end{matrix}$$

Where v₁=6.8×10⁻³±1.1×10⁻⁹, v₂=−32.2±3.6×10⁻³, and v₃=0.01±1.8×10⁻⁷ for internal rotation; and w₁=−0.81±2.8×10⁻⁶, w₂=−0.003±1.3×10⁻⁷, w₃=−67.9±4.3×10⁻⁴, and w₄=−0.001±1.8×10⁻⁷ for external rotation (see FIGS. 6(C), 6(D)).

The inventors found that interactions between frontal and transverse planes (i.e. CT_{FT}) were negligible compared to the interactions between the sagittal and frontal plane (CT_{SF}) and the sagittal and transverse planes (CT_{ST}) and therefore the interactions between the frontal and transverse planes can be assumed to be zero (i.e. CT_{FT}=0).

To be able to use the ACL force model in Equation (4) for estimating in vivo ACL force during dynamic laboratory-based dynamic motor tasks, the model in Equation (4) is combined with a neuromusculoskeletal model of the lower limb. Using three-dimensional (3D) motion capture, ground reaction loads, and surface electromyography (EMG) data from laboratory testing of females performing a standardized drop-landing task, as well as previously validated neuromusculoskeletal model [8, 22, 23], to calculate knee muscle and intersegmental loading (i.e. F_{muscle}, M_{muscle}, F_{intersegment}, and M_{intersegmental}) and knee flexion angle θ. These parameters are then used to calculate F_{AD}, Mᵥₐᵣ, M_{valg}, M_{IR}, and M_{ER} (see Equations (7) and (8), below) to then calculate total in vivo ACL force from Equations (1)-(4) above.

The graphs of FIGS. 5-9 will now be explained in more detail.

With reference to FIG. 5, the uni-planar ACL forces are shown. In FIG. 5(A), a graph of ACL force in the sagittal plane F_{ACL}^{sag} vs. knee flexion angles θ at different knee anterior drawer forces F_{AD} is shown. Symbols at F_{AD}=0 and 100 N are experimental data from [19, 20]. The remaining symbols are experimental data from [21]. The continuous curves are the fits derived from Equation (2).

Turning to FIGS. 5(B) and 5(C), the ACL force in the frontal plane F_{ACL}^{front} vs. knee varus and valgus moments (Mᵥₐᵣ, M_{valg}, respectively) at different knee flexion angles θ is shown. The symbols represent experimental data from [20] and the continuous curves are fits derived from Equation (3).

Referring now to FIGS. 5(D) and 5(E), the ACL force in the transverse plane F_{ACL}^{trans} vs. knee internal and external rotation moments (M_{IR}, M_{ER}, respectively) at different knee flexion angles θ is shown. The symbols represent experimental data from [20] and the continuous curves are fits derived from Equation (4).

Turning to FIG. 6, graphs of total ACL force F_{ACL} vs. knee flexion angle θ are illustrated.

In FIG. 6(A), F_{AD} in response to combined anterior drawer force F_{AD} and varus moment Mᵥₐᵣ is shown. The symbols shown are experimental data from [19] and the continuous curve is the fit derived from Equations (1) and (5).

In FIG. 6(B), F_{ACL} in response to combined F_{AD} and valgus moment M_{valg} is shown. The open circle symbol is experimental data from [19], the remaining symbols are representative of experimental data from [21] and the continuous curves are the fit derived from Equations (1) and (5).

Turning to FIG. 6(C), F_{ACL} in response to combined F_{AD} and knee internal rotation moment M_{IR} is shown. Left-pointing triangle symbols are experimental data from [19], the remaining symbols are experimental data from and the continuous curves are the fits derived from Equations (1) and (6).

Finally, in FIG. 6(D), F_{ACL} in response to combined F_{AD} and knee external rotation moment M_{ER} is shown. Diamond symbols are experimental data from [19], the remaining symbols are experimental data from [21] and the continuous curves are the fits derived from Equations (1) and (6).

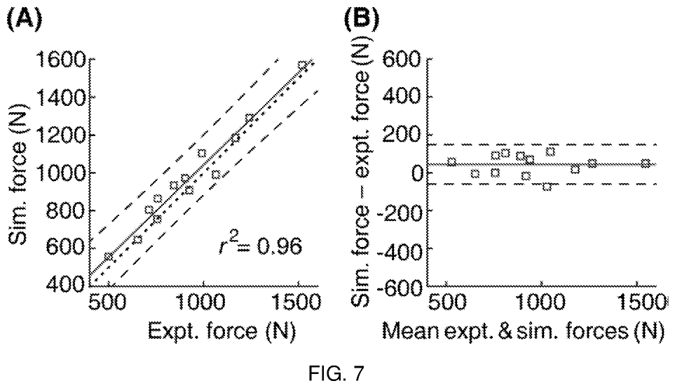

Turning now to FIG. 7, ACL force model validation statistics are shown.

With reference to FIG. 7(A), it can be seen that there is a comparison of cadaveric experimental ACL force (exponential force) and simulated ACL force (simulated force) derived from Equation (1). The solid line is the regression line (r²=0.96, RMSE=55.04N, P<0.001), which is not significantly different from the line of identity (the dotted line).

In FIG. 7(B), there is shown a Bland-Altman plot for the experimented and predicted ACL forces. The solid line represents mean difference (bias 44N and P=0.01), and the dashed lines are 95% limits of agreement (mean difference ±1.96 standard deviation, n=14 data points).

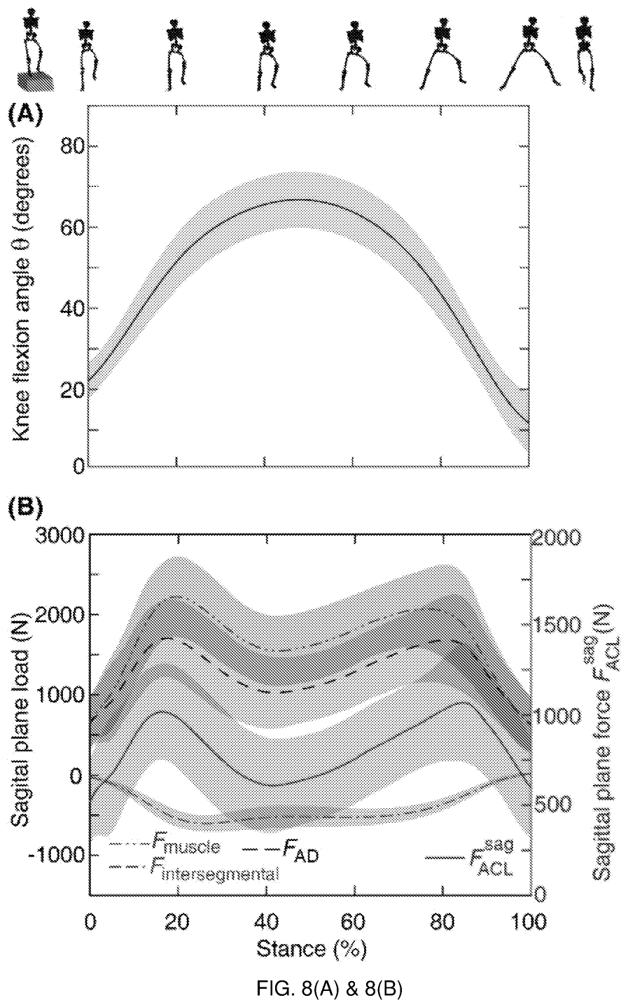

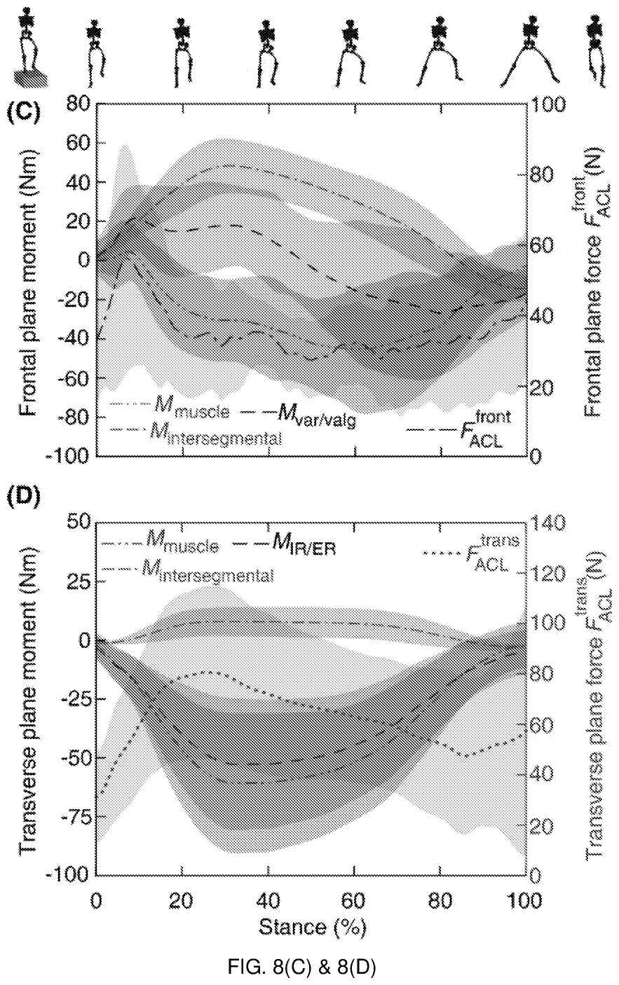

In FIG. 8, uni-planar ACL forces across the stance phase of the drop-landing tasks are modelled. With reference to the skeletal-type figure shown along the top of the schematic, this is representative of one participant during the stance phase of the task. The first (left-most) and last schematics (right-most) figures are before and after the stance, respectively, and are shown for clarity.

FIG. 8(A) shows knee flexion angle θ against stance. FIG. 8(B) shows sagittal plane knee loading and sagittal plane ACL force. On the left axis, muscle and intersegmental forces (F_{muscle} and F_{intersegmental}) obtained from the neuromusculoskeletal model, and net anterior drawer force F_{AD} are shown (see Equation (7)). On the right axis, ACL force in the sagittal plane F_{ACL}^{sag} obtained from Equation (2) is shown.

In FIGS. 8(C) and 8(D), frontal and transverse plane knee loadings and ACL forces are modelled. On the left axis, muscle and intersegmental moments (M_{muscle} and M_{intersegmental}), and net varus or valgus moment (Mᵥₐᵣ or M_{valg}), and internal or external rotation moments (M_{IR} or M_{ER}) are shown (see Equation (8)). On the right axis are ACL forces F_{ACL}^{trans} and obtained from Equations (3) and (4). The shaded regions show the standard deviation of the forces and moments. Directions of the sagittal plane knee load can be understood as follows: anterior draw (+) and posterior draw (−); frontal plane knee moment: varus (+) and valgus (−); transverse plane knee moment: internal rotation (+) and external knee rotation (−).

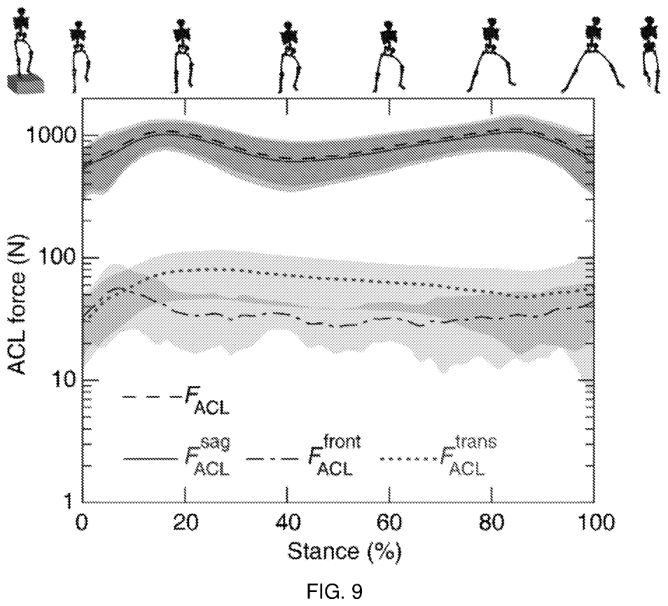

Turning now to FIG. 9, there is a graph of ACL force across stance phase of a drop-landing task. With reference to the skeletal-type figure shown along the top of the schematic, this is representative of one participant during the stance phase of the task. The first (left-most) and last schematics (right-most) figures are before and after the stance, respectively, and are shown for clarity.

ACL forces through each of the sagittal plane F_{ACL}^{sag}, the frontal plane F_{ACL}^{front}, and the transverse plane F_{ACL}^{trans} which contribute to total ACL force F_{ACL} are shown (see Equation (1)). The shaded regions show standard deviations of the forces.

In analysing the stance-phase of standardised drop-landing and lateral jump movement (see FIGS. 8(A)-(D)), the inventors found that ACL force was mainly driven through the sagittal plane, and due primarily to the action of muscles, rather than intersegmental loads—see FIG. 8(B).

In the frontal plane, contributions of muscle and intersegmental loads to ACL force are of opposite sign and of similar magnitude, resulting in small ACL forces—see FIG. 8(C).

In the transverse plane, muscle loading makes a greater contribution to ACL than intersegmental loading—see FIG. 8(D).

Observing FIG. 9, it can be seen that total ACL force F_{ACL} from multi-planar loading shows that force applied through the sagittal plane (F_{ACL}^{sag}) contributes significantly to F_{ACL} compared to force applied through the transverse and frontal planes, (F_{ACL}^{trans} and F_{ACL}^{front}). When ACL force reached its two local peaks, at approximately 17.5% and 80% of the stance phase of the drop-landing task, contribution through the sagittal plane to ACL force was above 94%, whereas contributions from the frontal and/or transverse planes are below 8% (see Table 1, below).

The first peak in ACL force occurs shortly after initial foot to ground contact (75±24 ms), which is comparable to analysis of cadaveric ACL rupture riming (54±24 ms) [24]. Notably, relative contributions of uni-planar forces do not sum to 100% due to the action of other articular soft tissues (i.e. ligaments and menisci) as well as rigid contact between femur and tibia, represented by the cross-terms in Equations (1), (5) and (6). This contrasts with existing models where the net ACL force has been formulated as the pure summation of the multiple uni-planar forces. As previously noted, this simple summation of multiple uni-planar ACL forces results over- and under-estimation of the total ACL force F_{ACL}.

The estimated total and uni-planar ACL forces are considered to be physiologically plausible on the basis that the estimations do not show any discontinuities or rapid fluctuations—see FIG. 9.

In addition, in contrast with calculations in [13], which used single participant data and modelled the lower bound ACL force in a drop-landing task (peak ACL force was ˜0.4 BW), the estimated total ACL force using the methods disclosed herein was below average failure loads for young ACL specimens which is approximately ˜2160N [25].

In [13], the predicted ACL force showed rapid fluctuations, where ACL force dropped to zero shortly after initial foot-to-ground contact, then increased sharply to its peak, and shortly after dropped back to zero. Such fluctuations in ACL force are not physiologically feasible during landing in the presence of high and continuous muscle forces. Thus, the inventors concluded that total ACL force is primarily generated through the sagittal plane (F_{ACL}^{sag}) primarily due to muscle loading (F_{muscle}). High muscle loading through the sagittal plane is due to anteriorly directed line of action of the quadriceps (via the patellar ligament) recruited to support the large external knee flexion and extension moments during landing and push-off phases, respectively. Furthermore, many knee spanning muscles possess lines of action creating tibiofemoral compression which contributes to net sagittal plane knee loading via the posteriorly sloped tibia.

TABLE 1
Loading Parameters at Peaks in ACL Force During a Drop-Landing for n =
13 participants (mean ± SD) - ACL: Anterior Cruciate Ligament; BW: Body Weight.

| | ACL | ACL | Timing of | Contributions to ACL force from | | |
|---|---|---|---|---|---|---|
| | Force | Force | ACL Force | uni-planar action (%) | | |
| Peak | (N) | (BW) | (% stance) | Sagittal | Frontal | Transverse |
| 1^{st} | 1137 ± 274 | 1.90 ± 0.57 | 17.53 ± 4.62 | 94.32 ± 2.36 | 7.99 ± 2.95 | 6.23 ± 2.52 |
| 2^{nd} | 1225 ± 301 | 2.03 ± 0.5 | 80 ± 9.13 | 95 ± 3.48 | 7.45 ± 5.21 | 4.07 ± 1.67 |

To develop and validate the ACL force model, two sets of experimental data [19-21] in which uni- and multi-planar external loads were applied to the cadaveric knee via a robotic rig were used. To calculate the contribution of muscles to knee loading, the cadaveric experiments of [21] were mimicked by implementing a musculoskeletal model in an OpenSim modelling environment. To simulate artificially-supplied quadricep and hamstring muscle forces (i.e. 1200N and 800N, respectively), it was assumed these forced were equally distributed among the individual muscles from each group (i.e. 300N for each quadricep and 200N for semitendinous, semimembranous, biceps femoris short head, and biceps femoris long head). The musculoskeletal model was then set to the reported knee posture of [21] by flexing the knee and tilting the pelvis-ground joint both by 25°. Since the cadaveric specimen was mounted upside down as in [21], the modelled pelvis was adjusted to be of minimal mass (i.e. 0.1 kg) and the sign for gravitational acceleration constant was changed from negative to positive. To incorporate the effects of the robotic loads applied to the knee in [21], compression and anterior drawer forces and varus/valgus and internal/external tibia rotation moments were applied to the musculoskeletal model. The cadaveric experimental muscle force contributions were also modelled by first calculating the muscle moment arms and lines of action. The muscle contributions to anterior drawer force, compression force, varus or valgus moment, and internal or external rotation moment were estimated by: muscle force (artificially supplied—see [21]) x muscle lines of action (or moment arms), depending on the plane of motion, defined relative to the tibia. From these muscle contributions, net loading in each plane of motion is calculated. In the sagittal plane, net anterior drawer force F_{AD} (Equation (7)) is:

F_{AD}=F_{muscle}+F_{intersegmental}+F_{contact}

where F_{muscle} is muscle force, F_{intersegmental} is intersegmental force representing the experimentally and robotically applied force (see [21]), and F_{contact} is knee joint contact force, which is the product of muscle compression onto a posteriorly sloped tibia.

In the frontal and transverse planes, respectively, net varus and valgus moments and net internal or external rotation moments (Equation (8)) are:

Mⱼ=M_{muscle}+M_{intersegmental}

where M_{muscle} and M_{intersegmental} are muscle and intersegmental moments for j=var/valg or IR/ER.

To unify measurement of ACL response across cadaveric data from [19-21], the measured ACL strain from [21] was converted to ACL force as: Force=(CSA×E×Strain), where for a typical ACL with linear elasticity, average ACL cross-sectional area (CSA) of approximately 65 mm² and Young modulus E˜113 MPa. Together, this transformed data is used in the development of the model described herein.

In validating the model, fourteen of the 25 multi-planar ACL force data points from [21], which were not included in the model development, were used to evaluate accuracy of the ACL force model described herein. Accuracy was assessed by RMSE, squared Pearson' correlation coefficient r², and Bland-Altman analysis—See

### In Vivo Experiment

An in vivo experiment involving healthy female adults performing a standardised drop-landing task in laboratory conditions at the Centre for Health, Exercise and Sports Medicine, University of Melbourne, Australia, was conducted to test the validity of the model described herein.

In the experiment, thirteen healthy (in that they had no known ACL damage or injury) female adults (age=22.99±2.57 years; mass=62.11±9.19 Kg; height=1.67±0.07 cm) completed at least three trials of the standardised drop-landing task unshod.

The task involved hopping down from a box (set at 30% of lower limb length) to land on one leg immediately followed by a 90° lateral jump landing on their opposite leg.

To measure the experiment, three-dimensional ground reaction loads were collected at 2400 Hz using ground-embedded force platforms (AMTI, Mass, USA), and kinematic data collected at 120 Hz using a 12-camera motion capture system (Vicon Motion Systems, Oxford, UK). The motion capture system measured 3D position of retroreflective markers placed on specific sites of the lower-limb and head-abdomen-trunk, as described in [39].

Wireless surface EMG sensors (Noraxon, Ariz., USA) were secured over the rectus femoris, vastus lateralis, vastus medialis, tibialis anterior, lateral gastrocnemius, medial gastrocnemius, lateral hamstrings, and medial hamstring muscles on the landing leg.

Sensors were placed according to Surface ElectroMyoGraphy for the Non-Invasive Assessment of Muscle (SENIAM) guidelines and EMG signals were recorded at 2400 HZ.

Marker trajectories and ground reaction data were filtered using a second-order, zero-lag Butterworth filter, with a low-pass cut-off frequency of 6 Hz.

The EMG data were band-pass filtered (between 30-300 HZ), full-wave rectified, and smoothed with a second-order Butterworth low-pass filter with a cut-off frequency of 6 HZ to produce linear envelopes. The EMG linear envelopes were then normalised to the maximum linear envelope value of the corresponding muscle from all available motion trials.

Musculoskeletal modelling was performed to calculate intersegmental joint moments and forces acting about planes of motion. For such, a generic 37 degree-of-freedom (DOF) full body model with 80 muscle tendon unit (MTU) actuators in the OpenSim musculoskeletal modelling environment was implemented. To calculate 6 generalised loads (moments and forces in each three planes of motion) at knee, ankle, and hip, the generic model was modified.

At the knee, dummy bodies of negligible mass/inertia and associated universal joints were added to the generic model topology. However, the original knee mobility, including flexion/extension with abduction/adduction, internal/external rotation, superior-inferior translation, and anterior/posterior translations prescribed as function of knee flexion were preserved.

At the ankle and hip, generic joints were expanded to 6 DOFs, but the newly expanded DOFs has zero mobility space. This means that the ankle mobility was restricted to plantar/dorsi-flexion, whereas hip mobilities were constrained to flexion/extension, adduction/abduction, and internal/external rotations.

This modified musculoskeletal model was linearly scaled to approximate participant mass and gross dimensions.

This scaling used prominent bony landmarks and hip joint centres. The hip joint centres were estimated using the Harrington regression equations. The scale factors were calculated as the quotient of the distance between specific pairs of experimental motion capture markers placed atop prominent anatomical landmarks and their corresponding model virtual markers. The marker pairs used to compute the scale factors to adjust width, height and depth of model bodies are shown in Table 2 below. In any dimensions, where multiple marker pairs are listed, the corresponding scale factor is an average of the scale factors calculated from each marker pair.

Following scaling, each MTUA's tendon slack and optimal fibre lengths were optimised to preserve the dimensionless force-length operating curves, as these are not preserved through linear scaling. Each muscle's maximum isometric strength was updated and implemented as performed previously in [32, 48], which estimates an individual's muscle volumes and length from their mass, height and limb length.

TABLE 2
Marker Pairs Used in Linear Scaling
of the Generic Template Model

Scaling Dimension

| Bodies | Width | Height | Depth |
|---|---|---|---|
| Trunk | NA | LASI-MAN | MAN-T10 |
| | | RASI-MAN | MAN-T2 |
| | | RASI-T10 | |
| | | LASI-T10 | |
| Pelvis | LASI-RASI | SACR-LHJC | RASI-SACR |
| | | SACR-RHJC | LASI-SACR |
| | | RASI-RHJC | |
| | | LASI-LHJC | |
| Femur | MEPI-LEPI | RHJC-MEPI | NA |
| | | RHJC-LEPI | |
| Shank | MMAL-LMAL | MEPI-MMAL | NA |
| | | LEPI-LMAL | |
| Foot | MT1-MT5 | NA | HEEL-MT5 |
| | | | HEEL-MT1 |

NA: scale factor of value 1 was used; LASI: left anterior superior iliac spine; RASI: right anterior superior iliac spine; MAN: jugular notch; T2: 2^{nd} thoracic vertebrae; T10: 10^{th} thoracic vertebrae; SACR: midpoint of right and left posterior superior iliac spine; LHJC: left hip joint centre; RHJC: right hip joint centre; MEPI: medical epicondyle of knee; LEPI: lateral epicondyle of knee; MMAL: medial malleolus; LMAL: lateral malleolus; MT1: 1^{st} metatarsal phalangeal joint; MT5: 5^{th} metatarsal phalangeal joint; HEEL: distal calcaneus.

The scaled musculoskeletal model used the laboratory data as inputs to determine angles, joint moments, and muscle kinematics. Inverse kinematics analysis was used to determine 3D joint angles, which were then combined with ground reaction data to run inverse dynamics analysis to determine model intersegmental joint loads (i.e. F_{intersegmental}, or M_{intersegmental}) for each DOF—see Equations (7) and (8). The OpenSim muscle analysis was then executed to determine MTU kinematics (i.e. instantaneous lengths, moment arms, and lines of action).

The forces for all lower-limb muscles during the drop-landing task were estimated using the calibrated EMG-informed neuromusculoskeletal modelling (CEINMS) toolbox. The CEINMS is a known OpenSim plug-in which uses EMG signals and MTU parameters to drive a Hill-type muscle model and predicts muscle excitations, muscle forces and joint moments. To verify the accuracy of muscle forces predicted by CEINMS, lower-limb joint moments generated by CEINMS predictions of muscle forces were compared to their corresponding inverse dynamics values obtained from OpenSim (r² are 0.99±0.01, 0.94±0.05, 0.93±0.04, and RMSE are 7.04±3.99, 11.32±6.22, 12.41±4.9 Nm for knee, hip and ankle, respectively).

This neuromusculoskeletal modelling approach predicts muscle and intersegmental loading (i.e. F_{muscle}, M_{muscle}, F_{intersegmental}, and M_{intersegmental} in Equations (7) and (8)) which were used in the ACL force model described herein.

Calculations of the total ACL force F_{ACL} calculated using the methods and systems disclosed herein have been validated by comparing the calculations with recent cadaveric experimental data [21] (see FIG. 7), and have been found to be highly accurate compared to other known methods. Root-mean-square error (RMSE) between cadaveric data and predicted ACL forces calculated using the methods disclosed herein is low (˜55N) and correlation is strong (r²=0.96)—see FIG. 7(A).

Bland-Altman analysis revealed good agreement cadaveric data and predicted ACL forces, with narrow limits of agreement ˜100N (12%) and negligible bias (˜44N) across loading magnitudes (see FIG. 7(B)). This agreement indicates that the ACL force model and calculations disclosed herein accurately estimate ACL forces in response to different knee loading magnitudes and combinations. Thus, in combination with a neuromusculoskeletal model of muscle dynamics, the ACL force model was found to be able to be used to accurately predict in vivo ACL loading.

Advantageously, the methods disclosed herein do not rely on explicit representations of anatomy and mechanical parameters of the knee's articular tissue. Rather, the teachings of the present disclosure are based on a set of algebraic expressions that provide real-time evaluation. This is particularly important as it enables ACL force to be used in biofeedback paradigms for injury prevention, training, and rehabilitation.

In addition, the present disclosure estimates muscle dynamics using neuromusculoskeletal modelling from biomechanical screenings, which combines subject- and task-specific empirical measurements of muscle excitations (e.g. electromyograms) and modelled musculotendon unit kinematics (i.e. lengths and moment arms).

Moreover, the teachings of the present disclosure provide a model that is developed and validated based on comprehensive cadaveric experimental data [19-21] across a wide range of ACL force magnitudes, which represent those observed in dynamic sporting tasks associated with ACL ruptures and everyday activities.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this disclosure can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, implementations of the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Implementations of the present disclosure can be realized in a computing system that includes a back end component, e.g., as a data server, or that includes a middleware component, e.g., an application server, or that includes a front end component, e.g., a client computer having a graphical user interface or a Web browser through which a user can interact with an implementation of the present disclosure, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this disclosure contains many specifics, these should not be construed as limitations on the scope of the disclosure or of what may be claimed, but rather as descriptions of features specific to particular implementations of the disclosure. Certain features that are described in this disclosure in the context of separate implementations can also be provided in combination in a single implementation. Conversely, various features that are described in the context of a single implementation can also be provided in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the present disclosure have been described. Other implementations are within the scope of the following claims. For example, the actions recited in the claims can be performed in a different order and still achieve desirable results.

In compliance with the statute, the invention has been described in language more or less specific to structural or methodical features. The term “comprises” and its variations, such as “comprising” and “comprised of” is used throughout in an inclusive sense and not to the exclusion of any additional features. It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

Throughout the specification and claims (if present), unless the context requires otherwise, the term “substantially” or “about” will be understood to not be limited to the value for the range qualified by the terms.

Any embodiment of the invention is meant to be illustrative only and is not meant to be limiting to the invention. Therefore, it should be appreciated that various other changes and modifications can be made to any embodiment described without departing from the spirit and scope of the invention.

### REFERENCES

The disclosure of each of the following documents is hereby incorporated in its entirety by reference:
1. Moffat K L, et al. (2008) Characterization of the structure-function relationship at the ligament-to-bone interface. Proc Natl Acad Sci USA 105(23):7947-7952.
2. Kiapour A M & Murray M M (2014) Basic science of anterior cruciate ligament injury and repair. Bone Joint Res 3(2):20-31.
3. Ajuied A, et al. (2014) Anterior cruciate ligament injury and radiologic progression of knee osteoarthritis: a systematic review and meta-analysis. Am J Sports Med 42(9):2242-2252.
4. Olsen O E, Myklebust G, Engebretsen L, & Bahr R (2004) Injury mechanisms for anterior cruciate ligament injuries in team handball: a systematic video analysis. Am J Sports Med 32(4):1002-1012.
5. Frobell R B, et al. (2008) The acutely ACL injured knee assessed by MRI: are large volume traumatic bone marrow lesions a sign of severe compression injury?Osteoarthritis Cartilage 16(7):829-836.
6. Viskontas D G, et al. (2008) Bone bruises associated with ACL rupture: correlation with injury mechanism. Am J Sports Med 36(5):927-933.
7. Quatman C E, Quatman-Yates C C, & Hewett T E (2010) A ‘plane’ explanation of anterior cruciate ligament injury mechanisms: A systematic review. Sports Med 40(9):729-746.
8. Lloyd D G, Buchanan T S, & Besier T F (2005) Neuromuscular biomechanical modeling to understand knee ligament loading. Med Sci Sports Exerc 37(11):1939-1947.
9. Maniar N, Schache A G, Sritharan P, & Opar D A (2018) Non-knee-spanning muscles contribute to tibiofemoral shear as well as valgus and rotational joint reaction moments during unanticipated sidestep cutting. Sci Rep 8(1):2501.
10. Kar J & Quesada P M (2012) A musculoskeletal simulation approach to studying anterior cruciate ligament strains and internal forces among young recreational women performing valgus inducing stop-jump activities. Ann Biomed Eng 40(8):1679-1691.
11. Kar J & Quesada P M (2013) A musculoskeletal modeling approach for estimating anterior cruciate ligament strains and knee anterior-posterior shear forces in stop-jumps performed by young recreational female athletes. Ann Biomed Eng 41(2):338-348.
12. Kernozek T W & Ragan R J (2008) Estimation of anterior cruciate ligament tension from inverse dynamics data and electromyography in females during drop landing. Clin Biomech (Bristol, Avon) 23(10):1279-1286.
13. Pflum M A, Shelburne K B, Torry M R, Decker M J, & Pandy M G (2004) Model prediction of anterior cruciate ligament force during drop-landings. Med Sci Sports Exerc 36(11):1949-1958.
14. Shelburne K B & Pandy M G (1997) A musculoskeletal model of the knee for evaluating ligament forces during isometric contractions. J Biomech 30(2):163-176.
15. Shelburne K B, Pandy M G, Anderson F C, & Torry M R (2004) Pattern of anterior cruciate ligament force in normal walking. J Biomech 37(6):797-805.
16. Shin C S, Chaudhari A M, & Andriacchi T P (2007) The influence of deceleration forces on ACL strain during single-leg landing: a simulation study. J Biomech 40(5):1145-1152.
17. Weinhandl J T, et al. (2013) Anticipatory effects on anterior cruciate ligament loading during sidestep cutting. Clin Biomech (Bristol, Avon) 28(6):655-663.
18. Weinhandl J T. et al. (2014) Reduced hamstring strength increases anterior cruciate ligament loading during anticipated sidestep cutting. Clin Biomech (Bristol, Avon) 29(7):752-759.
19. Markolf K L, et al. (1995) Combined knee loading states that generate high anterior cruciate ligament forces. J Orthop Res 13(6):930-935.
20. Markolf K L, Gorek J F, Kabi J M, & Shapiro M S (1990) Direct measurement of resultant forces in the anterior cruciate ligament. An in vitro study performed with a new experimental technique. J Bone Joint Surg Am 72(4):557-567.
21. Kiapour A M, et al. (2016) Strain Response of the Anterior Cruciate Ligament to Uniplanar and Multiplanar Loads During Simulated Landings: Implications for Injury Mechanism. Am J Sports Med 44(8):2087-2096.
22. Lloyd D G & Besier T F (2003) An EMG-driven musculoskeletal model to estimate muscle forces and knee joint moments in vivo. J Biomech 36(6):765-776.
23. Pizzolato C, et al. (2015) CEINMS: A toolbox to investigate the influence of different neural control solutions on the prediction of muscle excitation and joint moments during dynamic motor tasks. J Biomech 48(14):3929-3936.
24. Kiapour A M, et al. (2014) Timing sequence of multi-planar knee kinematics revealed by physiologic cadaveric simulation of landing: Implications for ACL injury mechanism. Clin Biomech 29(1):75-82.
25. Woo S L, Hollis J M, Adams D J, Lyon R M, & Takai S (1991) Tensile properties of the human femur-anterior cruciate ligament-tibia complex. The effects of specimen age and orientation. Am J Sports Med 19(3):217-225.
26. Herzog W & Read L J (1993) Lines of action and moment arms of the major force-carrying structures crossing the human knee joint. J Anatomy 182 (Pt 2):213-230.
27. Ward S H, Perraton L, Bennell K, Pietrosimone B, & Bryant A L (2019) Deficits in Quadriceps Force Control After Anterior Cruciate Ligament Injury: Potential Central Mechanisms. J Athl Training 54:414-417.
28. Meyer E G & Haut R C (2005) Excessive compression of the human tibio-femoral joint causes ACL rupture. J Biomech 38(11):2311-2316.
29. Fregly B J, Boninger M L, & Reinkensmeyer D J (2012) Personalized neuromusculoskeletal modeling to improve treatment of mobility impairments: a perspective from European research sites. J Neuroeng Rehabil 9:18.
30. Prilutsky B I & Zatsiorsky V M (2002) Optimization-based models of muscle coordination. Exercise and Sport Sciences Reviews 30(1):32-38.
31. Sauer T (2012) Numerical Analysis (Pearson Education, Boston, Mass.) 2nd Ed.
32. Rajagopal A, et al. (2016) Full-Body Musculoskeletal Model for Muscle-Driven Simulation of Human Gait. IEEE Trans Biomed Eng 63(10):2068-2079.
33. Delp S L, et al. (2007) OpenSim: open-source software to create and analyze dynamic simulations of movement. IEEE Trans Biomed Eng 54(11):1940-1950.
34. Kiapour A M, et al. (2015) Uni-directional coupling between tibiofemoral frontal and axial plane rotation supports valgus collapse mechanism of ACL injury. J Biomech 48(10):1745-1751.
35. Takeda Y, Xerogeanes J W, Livesay G A, Fu F H, & Woo S L (1994) Biomechanical function of the human anterior cruciate ligament. Arthroscopy 10(2):140-147.
36. Chandrashekar N, Mansouri H, Slauterbeck J, & Hashemi J (2006) Sex-based differences in the tensile properties of the human anterior cruciate ligament. J Biomech 39(16):2943-2950.
37. Altman D G & Bland J M (1983) Measurement in Medicine: The Analysis of Method Comparison Studies. J Roy Stat Soc D-SAT 32(3):307-317.
38. Sayer T A, et al. (2018) Effect of high and low-supportive footwear on female tri-planar knee moments during single limb landing. J Foot Ankle Res 11:51.
39. Schache A G & Baker R (2007) On the expression of joint moments during gait. Gait Posture 25(3):440-452.
40. Hermens H J, Freriks B, Disselhorst-Klug C, & Rau G (2000) Development of recommendations for SEMG sensors and sensor placement procedures. J Electromyogr Kinesiol 10(5):361-374.
41. Robertson D G & Dowling J J (2003) Design and responses of Butterworth and critically damped digital filters. J Electromyogr Kinesiol 13(6):569-573.
42. Saxby D J, et al. (2016) Tibiofemoral contact forces during walking, running and sidestepping. Gait Posture 49:78-85.
43. Hall M, Diamond L E, Lenton G K, Pizzolato C, & Saxby D J (2019) Immediate effects of valgus knee bracing on tibiofemoral contact forces and knee muscle forces. Gait Posture 68:55-62.
44. Walker P S, Rovick J S, & Robertson D D (1988) The effects of knee brace hinge design and placement on joint mechanics. J Biomech 21(11):965-974.
45. Harrington M E, Zavatsky A B, Lawson S E M, Yuan Z, & Theologis T N (2007) Prediction of the hip joint centre in adults, children, and patients with cerebral palsy based on magnetic resonance imaging. J Biomech 40(3):595-602.
46. Modenese L, Ceseracciu E, Reggiani M, & Lloyd D G (2016) Estimation of musculotendon parameters for scaled and subject specific musculoskeletal models using an optimization technique. J Biomech 49(2):141-148.
47. Ward S R, Smallwood L H, & Lieber R L (2005) Scaling of human lower extremity muscle architecture to skeletal dimensions (Abstract). ISB XYth Congress, p 502.
48. Handsfield G G, Meyer C H, Hart J M, Abel M F, & Blemker S S (2014) Relationships of 35 lower limb muscles to height and body mass quantified using MRI. J Biomech 47(3):631-638.
49. van Arkel R J, Modenese L, Phillips A T, & Jefers J R (2013) Hip abduction can prevent posterior edge loading of hip replacements. J Orthop Res 31(8):1172-1179.

1-23. (canceled)

24. A method of calculating in vivo force on an anterior cruciate ligament (ACL), the method comprising:
calculating a total load on an anterior cruciate ligament from an ACL force model defined by F_{ACL}=F_{ACL}^{sag}+F_{ACL}^{front}+F_{ACL}^{trans}+ΣⱼCTⱼ, wherein F_{ACL} is the total force on the F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT.

25. The method of claim 24, the method including:
measuring one or more biomechanical properties during a biomechanical screening task to obtain one or more biomechanical datum from the measured one or more biomechanical properties.

26. The method of claim 24, the method including:
monitoring one or more biomechanical properties of a subject performing a dynamic motor task;
generating one or more biomechanical datum from the monitoring of the one or more biomechanical properties of the subject performing the dynamic motor task;
receiving the one or more biomechanical screening datum as data inputs to a computer implemented ACL force model for calculating total load on an anterior cruciate ligament.

27. The method of claim 24, wherein
F_{ACL}^{sag}=a₁F_{AD}θ²+a₂F_{AD}θ+a₃F_{AD}+a₄e^{(a}^{₅}^{F}^{_{AD}}^{+a}^{₆}^{θ)}, wherein a₁=1.8×10⁻⁴±5.6×10⁻⁷, a₂=0.02±0.1×10⁻⁴, a₃=1.16±0.005, a₄=32.15±0.02, a₅=3.9×10⁻⁵±1.8×10⁻⁴, and a₆=−0.022±2.3×10⁻⁵, F_{AD} is anterior force drawer and θ is knee flexion angle;
$${F}_{ACL}^{front} = \{ \begin{matrix}{b}_{1} {M}_{var} {θ}^{2} + {b}_{2} {M}_{var} θ + {b}_{3} {M}_{var} + {b}_{4} {e}^{( {b}_{5} θ )} , if varus \\ {c}_{1} {M}_{valg} {θ}^{2} + {c}_{2} {M}_{valg} θ + {c}_{3} {M}_{valg} + {c}_{4} {e}^{( {c}_{5} θ )} + {c}_{6} {e}^{( {c}_{7} {M}_{valg} )} , if valgus\end{matrix} ,$$
wherein b₁=−0.0014±0.1×10⁻³, b₂=0.18±0.01, b₃=−6.8±0.21, b₄=23.85±2.03, b₅=−0.14±0.03 for varus moment; and c₁=−0.001±3.6×10⁻⁸, c₂=0.08±3.2×10⁻⁶, c₃=2.5±5.2×10⁻⁵, c₄=−3.3±0.6×10⁻⁵, c₅=−0.04±6.7×10⁻⁷, c₆=29.3±0.3×10⁻⁴, and c₇=0.02±3×10⁻⁷ for valgus moment, Mᵥₐᵣ is knee varus moment, M_{valg} is knee valgus moment and θ is knee flexion angle;
$${F}_{ACL}^{trans} = \{ \begin{matrix}{m}_{1} {M}_{IR} {θ}^{2} + {m}_{2} {M}_{IR} θ + {m}_{3} {M}_{IR} + {m}_{4} {e}^{( {m}_{5} θ )} , if internal rotation \\ {n}_{1} {M}_{E R} {θ}^{2} + {n}_{2} {M}_{E R} θ + {n}_{3} {M}_{E R} + {n}_{4} {e}^{( {n}_{5} θ )} , if external rotation\end{matrix} ,$$
wherein m₁=−0.005±2.4×10⁻⁷, m₂=0.63±0.2×10⁻⁴, m₃=−20.03±3.8×10⁻³, m₄=36.6±3.4×10⁻², m₅=−0.04±7.1×10⁻⁶ for internal rotation moment; and n₁=0.001±2×10⁻³, n₂=−0.16±0.02, n₃=7.8±0.4, n₄=23.3±2.5, n₅=−0.06±0.01 for external rotation moment, M_{IR} is internal rotation moment of the knee, M_{ER} is external rotation moment of the knee and θ is knee flexion angle;
$$C {T}_{SF} = \{ \begin{matrix}{p}_{1} {F}_{ACL}^{front} {e}^{( {p}_{2} {F}_{ACL}^{sag} )} + {p}_{3} θ {e}^{( {p}_{4} θ )} , if varus \\ {q}_{1} {e}^{( {q}_{2} {F}_{ACL}^{front} )} + {q}_{3} θ {e}^{( {q}_{4} θ )} , if valgus\end{matrix} ,$$
wherein p₁=−0.84±8.2×10⁻⁶, p₂=−0.004±6.9×10⁻⁸, p₃=2.9±1.3×10⁻⁵, and p₄=−0.041±1.02×10⁻⁷ for varus moment; and q₁=39.1±1.4×10⁻⁴, q₂=0.002±9.7×10⁻¹⁰, q₃=8.7±1.9×10⁻⁶, and q₄=−0.03±3.4×10⁻⁹ for valgus moment;
$$C {T}_{S T} = \{ \begin{matrix}{v}_{1} {F}_{ACL}^{sag} {F}_{ACL}^{trans} + {v}_{2} {e}^{( {v}_{3} θ )} , if internal rotation \\ {w}_{1} {F}_{ACL}^{trans} {e}^{( {w}_{2} {F}_{ACL}^{sag} )} + {w}_{3} {e}^{( {w}_{4} θ )} , if external rotation\end{matrix} ,$$
wherein v₁=6.8×10⁻³±1.1×10⁻⁹, v₂=−32.2±3.6×10⁻³, and v₃=0.01±1.8×10⁻⁷ for internal rotation; and w₁=−0.81±2.8×10⁻⁶, w₂=−0.003±1.3×10⁻⁷, w₃=−67.9±4.3×10⁻⁴, and w₄=−0.001±1.8×10⁻⁷ for external rotation; and CT_{FT}=0.

28. The method of claim 25, wherein the step of monitoring one or more biomechanical properties of a subject performing a dynamic motor task, further includes the subject wearing a first pair of shoes and the total load on the anterior cruciate ligament of the subject performing the dynamic motor task is a first total load; and the method further including:
monitoring one or more biomechanical properties of the subject performing the dynamic motor task, wherein the subject is unshod;
generating a second set of one or more biomechanical datum from the monitoring of the one or more biomechanical properties of the subject performing the dynamic motor task;
receiving the second set of one or more biomechanical screening datum as data inputs to a computer implemented ACL force model for calculating total load on an anterior cruciate ligament;
calculating a second total load on an anterior cruciate ligament of the subject performing the dynamic motor task from the computer implemented ACL force model, wherein the ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{trans} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT.

29. The method of claim 28, the method further including calculating a difference between the first total load on the anterior cruciate ligament of the subject performing the dynamic motor task and the second total load on the anterior cruciate ligament of the subject performing the dynamic motor task.

30. The method of claim 25, wherein the step of monitoring one or more biomechanical properties of a subject performing a dynamic motor task, further includes the subject wearing a first pair of shoes and the total load on the anterior cruciate ligament of the subject performing the dynamic motor task is a first total load; and the method including:
generating a second set of one or more biomechanical datum from the monitoring of the one or more biomechanical properties of the subject performing the dynamic motor task;
receiving the second set of one or more biomechanical screening datum as data inputs to a computer implemented ACL force model for calculating total load on an anterior cruciate ligament;
calculating a second total load on an anterior cruciate ligament of the subject performing the dynamic motor task from the computer implemented ACL force model, wherein the ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT.

31. The method of claim 30, the method further including calculating a difference between the first total load on the anterior cruciate ligament of the subject performing the dynamic motor task and the second total load on the anterior cruciate ligament of the subject performing the dynamic motor task.

32. A system for calculating an in vivo force on an anterior cruciate ligament (ACL), the system comprising:
a biomechanical screening system configured for a subject to perform a biomechanical screening task comprising a dynamic motor task, the biomechanical screening system comprising one or more biomechanical property monitoring apparatus for monitoring one or more biomechanical properties of the subject performing the dynamic motor task, wherein the one or more biomechanical monitoring apparatus generate one or more biomechanical datum; and
a computer having one or more electronic processors and a software product installed thereon, the software product being configured to operate the one or more electronic processors of the computer to calculate total load on an anterior cruciate ligament (ACL) from an ACL force model by:
receiving the one or more biomechanical datum as data inputs; and
calculating, via operation of the one or more electronic processors, total load on an anterior cruciate ligament using the ACL force model and the data inputs from the one or more biomechanical datum as inputs to the ACL force model,
wherein the ACL force model is defined by F_{ACL}=F_{ACL}^{sag}+F_{ACL}^{front}+F_{ACL}^{trans}+ΣⱼCTⱼ, wherein F_{ACL} is the total force on the ACL, F_{ACL}^{sag} is the force on the ACL in a sagittal plane, F_{ACL}^{front} is the force on the ACL in the frontal plane, F_{ACL}^{trans} is the force on the ACL in the transverse plane, and CTⱼ is the ACL force relationships in the sagittal-frontal (SF), sagittal-transverse (ST), and frontal-transverse (FT) planes, where j=SF, ST, FT.

33. The system of claim 32, the software product being configured to generate a graphical representation of the calculated total load on a display of the computer.

34. The system of claim 32, wherein the dynamic motor task comprises a drop-landing test.

35. The system of claim 34, the one or more biomechanical property monitoring apparatus of the biomechanical screening system comprising at least one of the following:
at least one electromyograph (EMG) sensors for attaching to the subject;
a motion capture system comprising a plurality of motion capture cameras and a plurality of retroreflective markers for attaching to the subject, wherein the plurality of motion capture cameras are configured to track the retroreflective markers; or
at least one ground embedded force platform configured to measure three-dimensional ground reaction loads of the subject.

36. The system of claim 34, the one or more biomechanical property monitoring apparatus of the biomechanical screening system comprising:
at least one electromyograph (EMG) sensors for attaching to the subject;
a motion capture system comprising a plurality of motion capture cameras and a plurality of retroreflective markers for attaching to the subject, wherein the plurality of motion capture cameras are configured to track the retroreflective markers; and
at least one ground embedded force platform configured to measure three-dimensional ground reaction loads of the subject.

37. The system of claim 36, wherein marker trajectories of the retroreflective markers are filtered by a second-order, zero-lag Butterworth filter having a low-pass cut-off frequency of 6 Hz.

38. The system of claim 36, wherein ground reaction data from the ground embedded force platform is filtered by a second-order, zero-lag Butterworth filter having a low-pass cut-off frequency of 6 Hz.

39. The system of claim 36, wherein signals from the EMG sensors are filtered by a band-pass filter (between 30-300 HZ), full-wave rectified, and smoothed with a second-order Butterworth low-pass filter with a cut-off frequency of 6 HZ generating a plurality of EMG linear envelopes.

40. The system of claim 39, wherein the EMG linear envelopes are normalised to the maximum linear envelope value of a corresponding muscle.

41. The system of claim 32, the one or more biomechanical property monitoring apparatus of the biomechanical screening system comprising a motion capture system.

42. The system of claim 41, the motion capture system comprising one or more of:
a plurality of inertial measurement units;
an electromagnetic measurement system; and
an Artificial Intelligence based system.
